(19) United States
(12) Patent Application Publication
(10) Pub. No.: US 2018/0195060 A1
(43) Pub. Date: Jul. 12, 2018

(54) METHOD FOR CONSTRUCTING LONG FRAGMENT DNA LIBRARY

(71) Applicants: BGI SHENZHEN, Shenzhen (CN); BGI SHENZHEN CO., LIMITED, Shenzhen (CN)

(72) Inventors: Ou WANG, Shenzhen (CN); Cankun CHANG, Shenzhen (CN); Lin LIN, Shenzhen (CN); Hui JIANG, Shenzhen (CN); Wenwei ZHANG, Shenzhen (CN)

(73) Assignees: BGI SHENZHEN, Shenzhen (CN); BGI SHENZHEN CO., LIMITED, Shenzhen (CN)

(21) Appl. No.: 15/567,963

(22) PCT Filed: Apr. 14, 2016
(86) PCT No.: PCT/CN2016/079278

§ 371 Date: Oct. 19, 2017

(30) Foreign Application Priority Data
Apr. 20, 2015 (CN) 201510187820.0

(51) Int. Cl. C12N 15/10; C40B 50/06; C40B 40/06

(52) U.S. Cl. CPC C12N 15/1093; C40B 40/06; C40B 50/06

(57) ABSTRACT

Disclosed is a method for constructing a long fragment DNA library, comprising the following steps: 1) breaking a long fragment DNA into target fragments of 3-10 kb by transposase, then amplifying the target fragments, and obtaining target fragment amplification products containing dUTP; 2) amplifying the dUTP in the products by removing the target fragments, fragmenting the target fragments secondarily into DNA short fragments of 300-1200 bp; 3) connecting both ends of the DNA short fragments with sequencing linker single chains A and sequencing linker single chains B respectively; and obtaining connecting sequencing linker products; and 4) PCR amplifying the connecting sequencing linker products, to obtain amplification products.

### TECHNICAL FIELD

The present invention relates to the field of biotechnology and, more particularly, to a method for constructing long fragment DNA library.

### BACKGROUND

Long Fragment Read (LFR), a DNA library constructing and sequencing technique (Methods and compositions for long fragment read sequencing, U.S. Pat. No. 8,592,150), is proposed by Complete Genomics, Inc. Long DNA fragments from the male parent and the female parent are physically separated by 384-well plates for genomic samples, and a library is constructed by adding different tag sequences. After the sequencing is completed, the genome is completely phased out, and whether mutation sites are on the same parent chromosome are confirmed. During the construction of the library, MDA amplification is performed on the long DNA fragment separated into the plate, and dUTP or other dNTP analogues are incorporated during the amplification process. The dNTP analog is subsequently removed by the action of endonuclease and the amplified product is interrupted into smaller fragments by a nick translation method and then a ligation operation of adapter A is achieved by a multi-step reaction via “ligating adapter 1-extension reaction-ligating adapter 2” using directional adapters. After the above steps are completed, cyclization is carried out and a sequencing adapter B is ligated according to the subsequent flow of the CG library construction to complete the library construction. The long fragment read technique can effectively improve the accuracy of sequencing and reduce the amount of starting DNA.

In order to meet the requirement of the library construction experiment, the DNA fragments separated into the 384-well plates need to be first amplified. Complete Genomics, Inc. amplificating the long DNA fragments separated into each cell of the 384-well plates by Multiple Displace Amplification (MDA). However, the MDA method tends to form non-specific amplification, and the single strand replaced in the amplification process will be combined with new random primers to form a higher complex structure to affect the subsequent reaction. Moreover, the multi-step enzyme reactions in the ligation process of adapter A are complicated and cumbersome.

### SUMMARY OF THE INVENTION

It is an object of the present invention to provide a method for constructing a long fragment DNA library.

The method provided by the invention comprises the following steps:

1) A long fragment DNA is subjected to cleavage by transposase, amplification to introduce dUTP and then removing the dUTP to obtain cleaved fragments;

2) Sequencing adapter single strands A with different tags and sequencing adapter single strands B with different tags which are partially complementary thereto, are added respectively in single strand form into a system containing the cleaved fragments to generate a reaction, so that the cleaved fragments are ligated to sequencing adapters at both ends, to obtain products ligated to different sequencing adapters by the combination of the tags in the sequencing adapter single strands A and sequencing adapter single strands B, wherein the cleaved fragments are corresponding to different sequencing adapters from each other;

the sequencing adapter single strands A with different tags and the sequencing adapter single strands B with different tags can be annealed to form the sequencing adapters;

3) PCR amplification is carried out with DNA matched to the sequencing adapters as primers while using the product after the ligation of the sequencing adapters as a template, and the PCR amplification product obtained is a PCR amplification product ligated to different sequencing adapters;

4) Constructing a library using the PCR amplification product ligated to different sequencing adapters to obtain a long fragment DNA library.

The constructing a library (see specifically the examples) comprises steps in turn of digesting dUTP of the PCR amplification product ligated to sequencing adapters, double-stranded cyclization, EcoP15 digestion, terminal repairing, dephosphorylation, ligating a second adapter, amplification of the product after ligating a second adapter, and isolating to obtain single strands, and single strand cyclization to obtain the long fragment DNA library.

In the above method, the step 1) of the method comprises the steps of:

(1) cleaving the long fragment DNA with a transposase and ligating amplification adapters at both ends of the fragment after cleavage to obtain a product ligated to the amplification adapters;

(2) performing a first PCR amplification with the product ligated to the amplification adapters as a template and the DNA matched to the amplification adapters as primers to obtain a first PCR amplification product; and dUTP is incorporated during the first PCR amplification process;

the PCR amplification reaction system (no template) comprises: 2× buffer 291.2 uL, Primer B (20 uM) 8.48 uL, Primer C (20 uM) 8.48 uL, dNTP (25 mM each) 20.17 uL, dUTP (4 mM) 5.04 uL, Pfu turbo Cx polymerase 7.68 uL, 20% (by volume) Triton X-100 aqueous solution 20.8 uL, 10% (by volume) Tween 20 aqueous solution 20.8 uL, adding nuclease free water to a total volume of 560 uL.

(3) removing the dUTP in the first PCR amplification product, obtaining a gap, and performing a gap translation, and adding A at the 3′end, and obtaining the product of the cleaved fragments as step 1).

In the above method, the transposase is a transposase that embeds an amplification adapter;

and/or, the amplification adapter is one or two types, the amplification adapter being formed by a transposase-recognized single-stranded DNA molecule and a single-stranded DNA molecule partially reverse complementary thereto;

and/or, the size of the fragment between the two adjacent action sites of the transposases that embed an amplification adapter is 3-10 kb.

and/or, in step (2) the DNA matched to the amplification adapters as primers means that, in the amplification adapter ligated to both ends of the fragment after cleavage, and further in the single-stranded DNA molecule reverse complementary to the transposase-recognized single-stranded DNA molecule, the primer pair formed by the remaining part of the sequence, except the reverse complementary sequence of the transposase-recognized single-stranded DNA molecule.

In the above method,

the amplification adapters are adapter 1 and adapter 2,

the amplification adapters are adapter 1 and adapter 2, wherein the adapter 1 is composed of a transposase-recognized single-stranded DNA molecule A and a single-stranded DNA molecule B partially reverse complementary thereto, and the adapter 2 is composed of the transposase-recognized single-stranded DNA molecule A and a single-stranded DNA molecule C partially reverse complementary thereto;

the DNA matched to the amplification adapters as primers are composed of primer B and primer C, wherein the primer B is the remaining sequence of the single-stranded DNA molecule B excluding the portion complementary to the transposase-recognized single-stranded DNA molecule A; the primer C is the remaining sequence of the single-stranded DNA molecule C excluding the portion complementary to the transposase-recognized single-stranded DNA molecule A;

and/or, the method for removing the dUTP in the first PCR amplification product comprises the following:

the first PCR amplification product is subjected to an enzymatic cleavage reaction using uracil DNA glycosylase and human apurinic/apyrimidinic endonuclease to obtain a cleavage product; and then the digested product is subjected to a polymerization reaction using polymerase I, Taq polymerase and dATP to obtain a DNA fragment having a size of 300 to 1200 bp.

The above enzyme digestion system (without template) comprises: UDG (2 U/uL) 14.56 uL, APE 1 (10 U/uL) 2.9 uL, adding nuclease free water to total volume of 560 uL.

The above polymerization reaction system (no template) comprises: Polymerase I (10 U/uL) 2.86 uL, Taq polymerase (5 U/uL) 5.7 uL, dATP (100 mM) 50.4 uL, adding nuclease free water to total volume of 560 uL.

In the above method, the tag sequences are obtained by arranging n bases, wherein the bases are at least one of A, G, C, and T, and n is equal to or greater than 8.

In the above method, the sequencing adapter single strands A with different tags comprises a fragment A, a fragment B, a tag sequence and a fragment C in the direction of 5′ to 3′.

The sequencing adapter single strands B with different tags comprises, in the direction of 5′ to 3′, a fragment reverse complementary to the fragment C, a tag sequence, a fragment D and a fragment reverse complementary to the fragment A;

In step 3), one primer in said primers is matched (forward or reverse complementary) to the fragment B in the sequencing adapter single strands A; and

the other primer in said primers is matched (reverse or forward complementary) to the fragment D in the sequencing adapter single strands B.

In the above method, the number of the sequencing adapter single strands A with different tags is less than or equal to 72, and the tag sequence of each single strand is different from each other;

the number of the sequencing adapter single strands B with different tags is less than or equal to 72, and the tag sequence of each single strand is different from each other;

n is greater than or equal to 8 and less than 15 in the tag sequence.

In the above method, in step 2), the sequencing adapter single strands A with tags and the sequencing adapter single strands B with different tags which are complementary thereto are added respectively in single strand form into a system containing the cleaved fragments to generate a reaction, comprises the following steps:

2)-A, 72 sequencing adapter single strands A with different tags are added respectively into 72 parallel lanes of a chip containing the cleaved fragments; the reaction buffer of the sequencing adapter single strands A does not contain T4 Ligase;

2)-B, 72 sequencing adapter single strands B with different tags are added respectively into 72 transverse lanes of the chip after the treatment of step 2)-A, to react to generate a product ligated to the sequencing adapter. The reaction buffer of the sequencing adapter single strands B contains T4 Ligase.

In the above method, step 3), one of the primers is identical to or reverse complementary to the fragment B in the sequencing adapter single strands A;

and the other primer is reverse complementary or identical to the fragment D in the sequencing adapter single strands B.

The PCR amplification is carried out by mixing the products ligated to different sequencing adapter in each well in the 5184 well plate for amplification;

In the above method, step (2), (3) in step 1) and step 2) of the method are carried out in a 5184-well chip. In the case where the cleaved fragments are divided into a plurality of portions, it is meant that the cleaved fragments are dispensed into each well of a 5184-well chip, and can be dispensed in step 1) or step 2).

In the above method, the long fragment DNA is a fragment of more than 100 kb. Specifically a fragment of 400 kb;

The transposase is a Tn5 transposase; in this example, the transposase embedding the amplification adapters is a product of Vazyme, named TruePrep mini DNA Sample Prep Kit (S302-01-B) Transposase Kit, the best concentration for use is 100 times dilution of the transposase. The amount of the transposase embedding the amplification adapters and the long fragment DNA are shown in the following reaction system. The reaction system of the above reaction includes the single-stranded DNA molecule B, the single-stranded DNA molecule C, a reaction buffer, dNTP, dUTP and polymerase; 2 μL transposase embedding the amplification adapters of 100 times dilution; 2 uL 5× buffer (S302-01, Vazyme), 7 ng of human genomic DNA (length greater than 100 kb), and adding water to 10 μL.

In the above method, step (2), (3) in step 1) and step 2) of the method are carried out by wafergen MSND pipetting platform to add various substances to the wells of the chip.

In the above method, after adding various substances to the wells of the chip by wafergen MSND pipetting platform, the method further comprises the steps of centrifuging the substances.

In the above method, steps (1) and (2) of step 1) further comprise the steps of: digesting the transposase with a denaturing agent; the denaturing agent is specifically 0.1-1% SDS solution; and the digestion conditions are carried out at 25° C. for 10 minutes.

The nucleotide sequence of the transposase-recognized single-stranded DNA molecule A is described in SEQ ID NO: 5 in the Sequence Listing;

The nucleotide sequence of the single-stranded DNA molecule B is described in SEQ ID NO: 6 in the Sequence Listing;

The nucleotide sequence of the single-stranded DNA molecule C is described in SEQ ID NO: 7 in the Sequence Listing;

The nucleotide sequence of the primer B is described in SEQ ID NO: 12 in the Sequence Listing;

The nucleotide sequence of the primer C is described in SEQ ID NO: 13 in the Sequence Listing;

The nucleotide sequence of the sequencing adapter single strands A is described in SEQ ID NO: 1 or 8 in the Sequence Listing;

The nucleotide sequence of the sequencing adapter single strands B is described in SEQ ID NO: 2 or 9 in the Sequence Listing;

The nucleotide sequence of the single strand primer A (one primer) is described in SEQ ID NO: 3 or 10 in the Sequence Listing;

The nucleotide sequence of the single strand primer B (the other primer) is described in SEQ ID NO: 4 or 11 in the Sequence Listing.

In the above method, step (1) and (2) of step 1) further comprise the steps of: digesting the transposase with a denaturing agent; the denaturing agent is specifically 0.1-1% SDS solution; and the digestion conditions are carried out at 25° C. for 10 minutes. The denaturing agent is an SDS solution, more specifically comprises 11.2 uL of 1% SDS (mass/volume percent) and 548.8 uL 2× buffer (MP01137, Complete Genomics).

In the above method, in step (1) of step 1), the conditions of the transposase cleavage reaction are at 55° C. for 5 minutes;

in step (2) of step 1), the annealing temperature of the amplification is at 68° C. and the annealing time is 18 minutes;

in step (3) of step 1), the conditions of the digestion reaction are at 37° C. for 2 hours and then at 65° C. for 15 minutes; the conditions of the polymerization conditions are at 23° C. for 1 hour and then at 65° C. for 30 minutes;

in step 2)-B of step 2), the reaction conditions are at 20° C. for 2 hours;

in step 3), the annealing temperature of the PCR amplification is 68° C. and the annealing time is 10 min.

The long fragment DNA library prepared by the above method is also a range of protection of the present invention.

It is another object of the present invention to provide a method for preparing a product ligated to sequencing adapters for the construction of a long fragment DNA library.

The method provided by the present invention comprising the steps of: step 1) to 2) in the above method.

The use of the above methods in constructing a long fragment DNA library is also a scope of protection of the present invention.

The denaturing agents of the present invention may be NT buffer, SDS or other agents that can denature the transposase and detach the same from the DNA long fragment.

### DESCRIPTION OF THE DRAWINGS

FIG. 1 shows a schematic diagram of the transposase interrupting a genomic fragment.

FIG. 2 shows a schematic diagram of the chip of wafergen MSND pipetting platform.

FIG. 3 shows a schematic diagram of the transposase combining with genomic DNA.

FIG. 4 shows the amplification with primer sequences that match the adapter 1/2 inserted by the transposase after the transposase is detached.

FIG. 5 shows dUTP cleavage from the PCR product under the action of UDG enzyme and APE1 enzyme and leaving a lbp gap on the product.

FIG. 6 shows a gap translation dependent on the digestion of dUTP.

FIG. 7 shows a schematic diagram of adding two single strands with a tag respectively of the first adapter.

FIG. 8 is a schematic diagram of ligation two single strands of the first adapter and an insert segment.

FIG. 9 is a schematic diagram of the PCR amplification reaction after the ligation of the first adapter.

FIG. 10 shows two spray mode diagram for wafergen MSND.

FIG. 11 shows an electrophoretic gel of the PCR product after interruption of fragments by transposase.

FIG. 12 shows an electrophoretic gel of the product in each step of the library construction by a chip.

FIG. 13 is a cleavage product digested by EcoP15 endonuclease.

FIG. 14 shows an electrophoretic gel after the single strand cyclization.

FIG. 15 is the histogram of the amount of data in each well and the corresponding frequency.

FIG. 16 is a sequencing depth distribution curve.

### BEST MODE FOR CARRYING OUT THE INVENTION

The experimental methods used in the following examples are conventional methods unless otherwise specified.

The materials, reagents and the like used in the following examples are commercially available, unless otherwise specified.

In the present invention, specific sequences are inserted by transposase, and PCR amplification is carried out by the specific sequences in a chip with 5184 wells, and the library is finally constructed.

In the following examples, by means of a wafergen company's MSND pipetting platform and a chip with 5184 wells, samples or reaction solutions can be dispensed in different ways each time, as shown in Table 1 below:

### Table 1 is Different Ways of Dispensing

| | Dispensing | |
|---|---|---|
| Dispensing program | volum | Application |
| Single sample dispensing | 35 nL | The same sample is dispensed |
| process | | into 5184 wells |
| 72 Samples 35 nL | 35 nL | A single sample is dispensed |
| Dispensing Process | | into a paralle lane of the chip |
| 72 Samples 50 nL | 50 nL | A single sample is dispensed |
| Dispensing Process | | into a transverse lane of the chip |

### Example 1: Construction of a Long Fragment DNA Library (for Complete Genomics Sequencing Platform)

First, the long fragment DNA was interrupted into a 3-10 kb target fragment by transposase

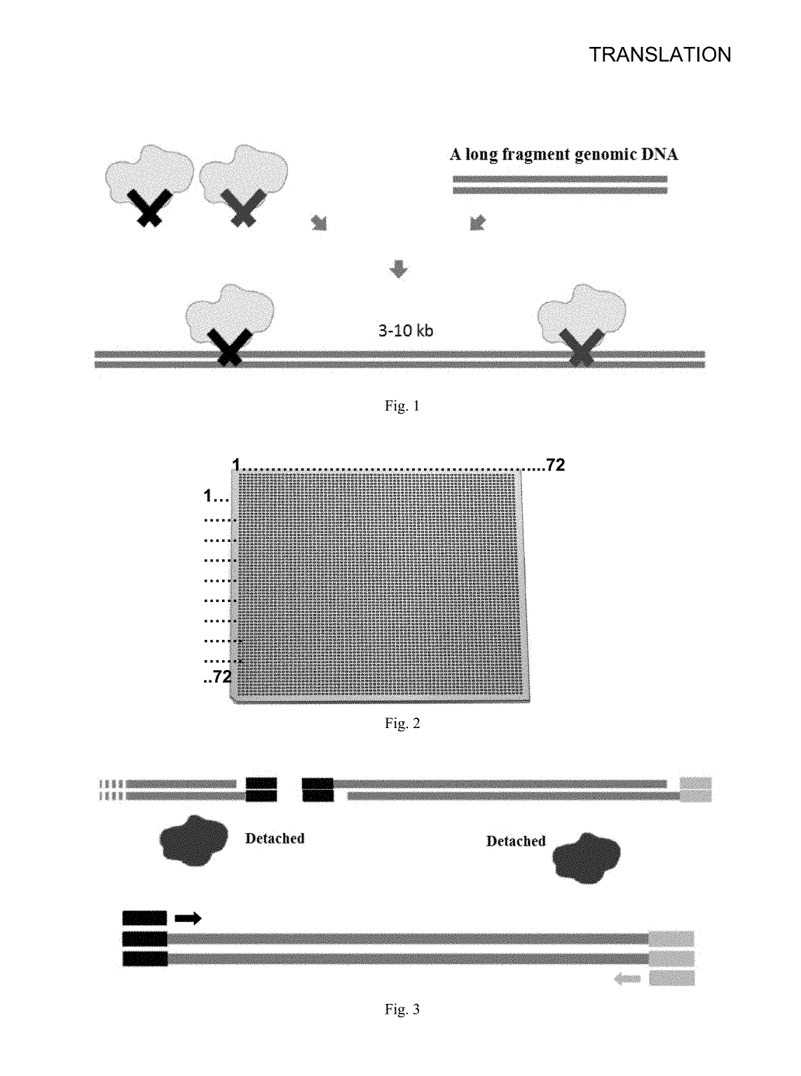

FIG. 1 shows a schematic diagram of the transposase interrupting a genomic fragment. The transposase embedding adapter 1 and adapter 2, after in combination with genomic DNA, random combining with locations of the genome, by controlling the amount of transposase used, control the size of the fragment between two adjacent transposase action sites to between 3 and 10 kb.

FIG. 2 shows a schematic diagram of the chip of wafergen MSND pipetting platform. The chip has a total of 72 transverse lanes, a total of 72 parallel lanes, and a total number of 5184 wells.

The transposase can embed 1 or 2 types of adapters.

The transposase embedding amplification adapters used in the present example has two kinds of adapters, that is, an adapter obtained from annealing a transposase-recognized single-stranded DNA molecule A and a single-stranded DNA molecule B partially reverse complementary thereto, and an adapter obtained from annealing a transposase-recognized single-stranded DNA molecule A and a single-stranded DNA molecule C partially reverse complementary thereto. The two adapters were mixed at equal concentrations of 100 uM and then mixed with the transposase and embedded. In this example, the transposase embedding the amplification adapters is a product of Vazyme, named TruePrep mini DNA Sample Prep Kit (S302-01-B) Transposase Kit.

The above sequence information is as follows:

Transposase-recognized single-stranded DNA mole-
cule A:
(SEQ ID NO: 5)
5′-CTGTCTCTTATACACATCT-3′;
Single-stranded DNA molecule B:
(SEQ ID NO: 6)
5′-TCGTCGGCAGCGTCAGATGTGTATAAGAGACAG-3′;
Single-stranded DNA molecule C:
(SEQ ID NO: 7)
5′-GTCTCGTGGGCTCGGAGATGTGTATAAGAGACAG-3′.

In a centrifuge tube, the amount of Tn5 transposase embedding adapters used is controlled to act on genomic DNA, leaving two adjacent transposase action sites from the same DNA fragment at a longer distance (about 3-10K). Due to the characteristics of the transposase itself, after the action of the transposase, it will not immediately detach from the DNA, but still exist in the action sites, connecting the interrupted long fragment DNA, so the entire DNA long fragment still maintain the original length rather than disconnected.

The above-mentioned transposonase products were diluted to a certain concentration (allowing sufficient separation between DNA long fragments), and then transferred to a chip using wafergen's micro-pipetting platform to achieve physical separation of DNA long fragments. The chip has 5184 wells, that is, a single DNA sample can be separated up into 5184 wells. The reagents added in the subsequent steps are carried out using the pipetting platform.

1. Exploring Transposase Concentration Used

Performing a PCR reaction by mediating with transposase, and fragment length of 3-10 kb required for the experiment was obtained.

The transposase embedding amplification adapters was diluted to 50 times, 100 times and 150 times, respectively.

The following reaction system was added to a PCR reaction tube: 5× buffer 2 uL (S302-01, Vazyme), human genomic DNA (genomic DNA extracted from isolated blood cells; 7 ng, greater than 100 kb) 6 uL, transposase (different dilution times) 2 uL.

The reaction systems with different dilution times transposase were mixed and then put into a PCR instrument, at 55 Celsius for 5 minutes and then cooled to room temperature. The reaction mixture was added a denaturing agent SDS to a final concentration of 0.1%. The mixture was mixed at room temperature and maintained for 10 minutes to obtain an target fragment of size of 3-10 kb. Then the target fragment size of 3-10 kb was diluted to a final concentration of about 8 pg/uL.

2) PCR Reaction

The target fragment size of 3-10 kb obtained in the above step 1) was subjected to PCR amplification by adding the following PCR reaction system to obtain an target fragment amplification product.

The primers used for amplification are, in the amplification adapter ligated to both ends of the fragment after cleavage, and further in the single-stranded DNA molecule reverse complementary to the transposase-recognized single-stranded DNA molecule, the primer pair formed by the remaining part of the sequence, except the reverse complementary sequence of the transposase-recognized single-stranded DNA molecule, specifically as follows:

(SEQ ID NO: 12)
Primer B: 5′-TCGTCGGCAGCGTC-3′
(SEQ ID NO: 13)
Primer C: 5′-GTCTCGTGGGCTCGG-3′

The above 100 uL PCR reaction system comprises: 5 uL of the target fragment size of 3-10 kb (8 pg/uL), 50 uL of 2× buffer, 0.5 uL of primer B (20 uM), 0.5 uL of primer C (20 uM), 0.5 uL of DNA polymerase, adding water to 100 uL.

The PCR procedure shown in Table 2 was used for amplification.

### Table 2 is a PCR Procedure

| Reaction temperature | Reaction time | | Number of cycles |
|---|---|---|---|
| 72° C. | 10 | min | |
| 94° C. | 3 | min | |
| 94° C. | 30 | sec | 18 cycles |
| 68° C. | 18 | min | |

| | |
|---|---|
| 10° C. | On hold |

The results of different dilutions of the transposase were as follows:

In the case of 100-fold dilution of the transposase, it can be seen that the amplification product of 3-10 kb was obtained as the target fragment.

In the case of 50-fold dilution of transposase and 150-fold dilution of transposase, no amplification product of 3-10 kb was obtained as the target fragment.

It can be seen that the optimal transposase dilution is 100 times dilution.

2. The Optimal Transposase Dilution Concentration Fragmented Genomic DNA Target Fragment

The genomic DNA was treated with the optimal 100-fold dilution of the transposase of the above-mentioned step 1, and the genomic DNA was ligated to PCR adapter sequences embedded in the transposase between about 3-10K, and the adapter sequences were used as the starting point for PCR amplification, as follows:

1) Fragmenting of DNA by Transposase

The transposase embedding amplification adapters and human genomic DNA were reacted according to the following reaction system, as follows:

The above reaction system was as follows: 2 uL of 100-fold diluted transposase embedding amplification adapters, 2 uL of 5× buffer (S302-01, Vazyme), 7 ng of human genomic DNA (length 400 kb), adding water to 10 uL.

The reaction system can be expanded or reduced in proportion.

The reaction was as follows: the reaction system was placed in a PCR apparatus at 55° C. for 5 minutes and then cooled to room temperature to give a reaction product which was diluted to a final concentration of about 8 pg/uL to give a diluted reaction product (genomic DNA inserted by transposase every 3-10 KD).

Using the wafergen MSND pipetting platform single sample dispensing process, the diluted fragmented product was partitioned into each well of the 5184 well plate (200 nl of each well) at 35 nL/well; the chip was centrifuged (4000 rpm, 5 min), and thus the dispensed liquid was deposited to the bottom of the chip to obtain a 5184-well plate containing the reaction product.

FIG. 3 shows after the transposase combining with the genomic DNA and inserting the adapters 1/2, it will not immediately detach but attach to its action site. A certain concentration of SDS was added to the reaction solution to render the transposase detach from the DNA.

2) Digesting Transposase

Using wafergen MSND pipetting platform single sample dispensing process, the denaturing reagent was added to each well of the 5184-well plate containing the reaction product obtained in step 1) above, and the chip was centrifuged (4000 rpm, 5 min) to allow the dispensed liquid to settle to the bottom of the chip; after centrifugation, maintained at room temperature for 10 minutes for digestion of the transposase, resulting the transposase detach from the genomic DNA and the realization of the fragmenting of genomic DNA, to obtain 3-10 KD target fragment, and the chip containing 3-10 KD target fragment is the 5184-well chip.

Other denaturing reagent and concentrations may be used in this procedure, and are not limited to SDS.

The above denaturing reagent consists of 11.2 uL of 1% SDS (mass/volume percent) and 548.8 uL of 2× buffer.

3) Amplifying the Target Fragment to Obtain the Target Fragment Amplification Product

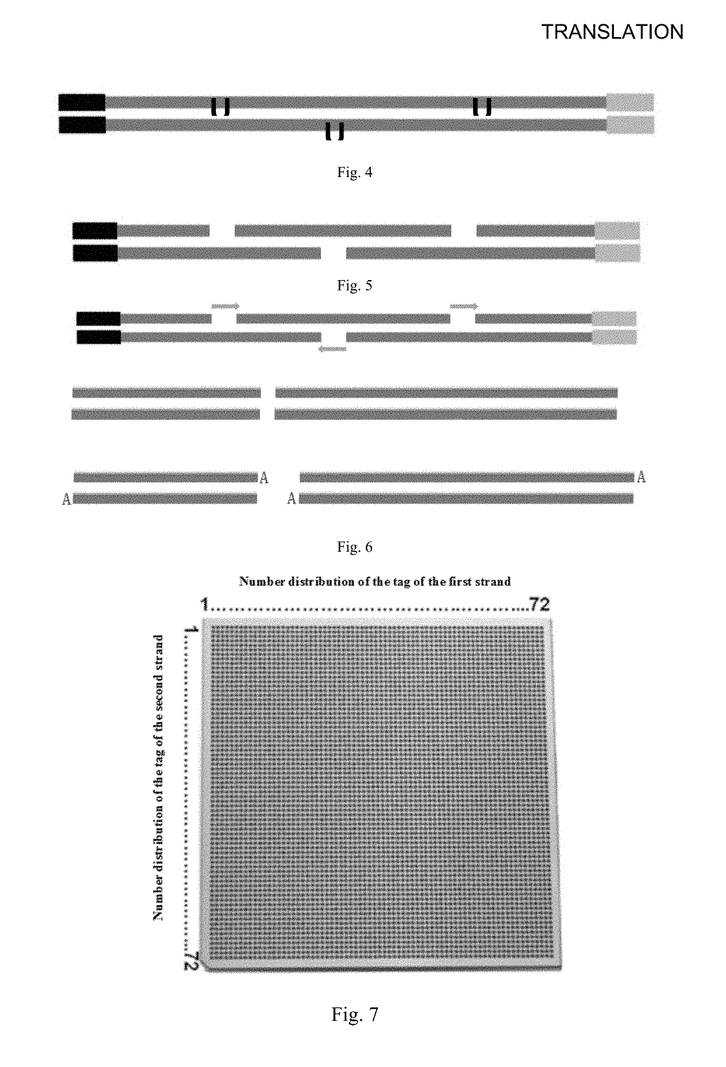

FIG. 4 shows introducing primer B and primer C into the PCR system to amplify after completion of the detaching of the transposase. At the same time, a certain proportion of dUTP (4%, dUTP: dATP+dTTP+dCTP+dGTP) was put into the PCR system, and a certain proportion of dUTP was incorporated into the amplification product.

Using designed primers, the product obtained from the previous step was subjected to PCR. The dUTP (dUTP: dNTP=4: 100) was introduced into the PCR system so that part of dTTP was replaced by dUTP in the final product, as follows:

Using wafergen MSND pipetting platform single sample dispensing process, the following PCR reaction system was added to each well of the 5184-well chip containing the 3-10 KD target fragment obtained in step 2). After centrifugation, the dispensed liquid was allowed to settle and the chip plate was placed in a PCR instrument for a PCR amplification reaction being carried out to obtain a 5184-well chip containing the dUTP in the amplification product of the target fragment.

A proportion of dUTP is added to the above reaction system so that a portion of the dTTP is replaced by dUTP in the amplification product, so that the amplification product can be interrupted again by dUTP removal at a later stage.

The PCR amplification reaction system described above comprises: 2× buffer 291.2 uL, single-stranded DNA molecule B (20 uM) 8.48 uL, single-stranded DNA molecule C (20 uM) 8.48 uL, dNTP (25 mM each) 20.17 uL, dUTP (4 mM) 5.04 uL, Pfu turbo Cx polymerase 7.68 uL, 20% (by volume) Triton X-100 aqueous solution 20.8 uL, 10% (by volume) Tween 20 aqueous solution 20.8 uL, adding nuclease free water to a total volume of 560 uL.

The above PCR amplification reaction procedure is shown in Table 3:

### Table 3 Shows the PCR Reaction Procedure

| Reaction temperature | Reaction time | | Number of cycles |
|---|---|---|---|
| 72° C. | 10 | min | |
| 94° C. | 3 | min | |
| 94° C. | 30 | sec | 18 cycles |
| 68° C. | 18 | min | |

| | |
|---|---|
| 10° C. | On hold |

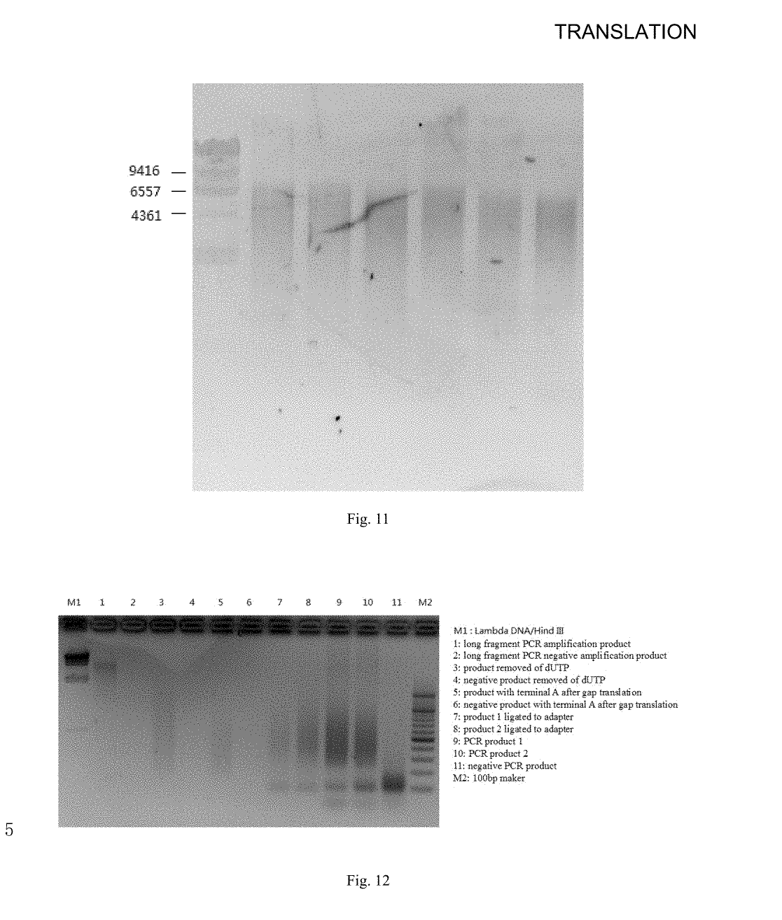

FIG. 11 shows an electrophoretic gel of the PCR product after interruption of fragments by transposase.

After completion of the reaction, the chip was placed in a 37° C. metal bath and allowed to maintain for 15.5 minutes to dry the excess liquid.

Second, performing a second fragmenting 3-10 KD target fragment into 300-1200 bp DNA short fragment

1) Removing dUTP to Form a Single Strand Gap

Using the enzyme (UDG, USER, etc.) with separate activity of removal of dUTP, treated the PCR product of previous step, to remove the dUTP and the sugar skeleton at that position to obtain a DNA double-stranded product with a gap of one base length (FIG. 5), as follows:

The dUTP introduced in the amplification process was digested using uracil DNA glycosylase (UDG) and human apurinic/apyrimidinic endonuclease (APE 1).

Specifically as follows:

The enzyme digestion system was added to each well of the 5184-well chip containing the target fragment amplification product including dUTP, according to the wafergen MSND pipetting platform single sample dispensing process, followed by centrifugation to allow the dispensed liquid to settle (4000 rpm, 5 min); then the chip was put into a dedicated PCR instrument to allow an enzyme digestion reaction, to give a 5184-well chip containing the enzyme digestion product.

The above enzyme digestion system comprises: UDG (2 U/uL) 14.56 uL, APE 1 (10 U/uL) 2.9 uL, adding nuclease free water to a total volume of 560 uL.

The above reaction conditions were as follows: 37° C. for 2 hours, 65° C. for 15 minutes, and then returned to room temperature.

2) Finishing Fragment by a Polymerization Reaction

The DNA is interrupted by gap translation and A is added at the end. DNA polymerase I and Taq enzymes were added to the reaction system. The product of the previous step was interrupted at 23° C. using the activity of the DNA polymerase I (5′-3′exonuclease activity and 5′-3′ polymerase activity). The DNA polymerase I binds to the gap left after removal of dUTP, and then DNA bases were excised from the 5′-3′direction and DNA bases were polymerized from the 5′-3′ direction to achieve a gap translation from 5′-3′direction. When the two gaps in the DNA double strand meet due to the translation, the DNA double strand breaks into smaller fragments. At the same time, in the system by adding Taq polymerase, at 65° C. to repair the DNA double strand to blunt, and a dATP base is added in the 3′end. At this temperature, DNA polymerase I is inactivated due to denaturation.

FIG. 6 shows the gap produced by the digestion of dUTP, and the DNA polymerase I which was subsequently put into the reaction system, binds to the gap position to exert its 5′-3′direction cleavage activity and 5′-3′ direction synthesis activity, thus the gap is translated in the direction of 5′-3′. In the double strand, the gaps located in the positive and negative strand at the same time to translate and meet, and ultimately break the entire double strand at the meeting position. While the Taq enzyme added to the system, exerts its activity at a temperature of 65° C., so that the 3′end of the product forms an A base.

Using the gap translation characteristic of the polymerase, the DNA fragment was interrupted by the gap translation after the removal of the dUTP, and the Taq polymerase was added to the reaction system, and thus dATP was added to the 3′end of the DNA product after the gap translation, specifically as follows:

The polymerization reaction system was dispensed into the wells of the 5184-well chip containing the step 1) digestion product, followed by centrifugation to allow the dispensed liquid to settle (4000 rpm, 5 min). And then the chip was put into a dedicated PCR instrument, to carry out a polymerization reaction to give a 5184-well chip containing 300-1200 bp DNA fragment.

The above polymerization reaction system comprises: Polymerase I (10 U/uL) 2.86 uL, Taq polymerase (5 U/uL) 5.7 uL, dATP (100 mM) 50.4 uL, adding nuclease free water to a total volume of 560 uL.

The above reaction conditions were 23° C. for 1 hour, 65° C. for 30 minutes, and then returned to room temperature.

Third, ligating sequencing adapters

Sequencing adapters with partial sequencing primers were ligated at both ends of the 300-1200 bp DNA fragment obtained in the above second step, and the sequencing adapters were obtained by annealing sequencing adapter single strands A with different tags and sequencing adapter single strands B with different tags. In order to distinguish each well on the chip in the process of sequencing, in the chip in each parallel lanes (corresponding to the sequencing adapter single strands A) adding the sequencing adapter single strands A with different tags numbered 1-72, and each transverse lanes (corresponding to the sequencing adapter single strands B) adding the sequencing adapter single strands B with different tags numbered 1-72, so that a matrix of 72×72 is formed by two separate tag sequences so that each well has a unique combination of double tag sequences.

Sequencing adapter single strands A:

(SEQ ID NO: 1)
CGAAGCACTCAAA [symbol] NNNNNNNNNNCAGT
ACG [symbol] *T;

where all the bold from 5′ to 3′ are as follows:

The fragment A ( [symbol] [symbol] ) is reverse complementary to [symbol] [symbol] at the 3′end of the sequencing adapter single strands B;

The fragment B (GGTCGCCAGCCCTATGGC) is identical to the first strand of the PCR primer shown in SEQ ID NO: 3, but only the U of the SEQ ID NO: 3 is replaced by T;

NNNNNNNNNN is a 10 bases tag sequence;

The fragment C (TCAGCAGT) is reverse complementary to [symbol] at the 5′end of the sequencing adapter single strands B;

* represents phosphorothioate, that is, when the last base is synthesized, a nucleotide of phosphorothioate is used.

Sequencing adapter single strands B:

(SEQ ID NO: 2)
/5Phos/ [symbol] GTCGAGANNNNNNNNNN [symbol] [symbol]
GAGTGCTTCGAA/3′AmMO/;

where all the bold from 5′ to 3′ are as follows:

[symbol] is reverse complementary to the fragment [symbol] at the 3′end of the sequencing adapter single strands A;

NNNNNNNNNN is a 10 bases tag sequence;

The fragment D ( [symbol] [symbol] [symbol] ) is reverse complementary to the second strand of the PCR primer shown in SEQ ID NO: 4, but only the U of SEQ ID NO: 4 is replaced by T;

GAGTGCTTCG is reverse complementary to the fragment A (CGAAGCACTC) at the 5′end of the sequencing adapter single strands A.

FIG. 7 shows a schematic diagram of adding two single strands with a tag respectively of the first adapter. In order to make each well in the chip with a unique tag sequence to distinguish between each other, the use of two tag sequences to form a combination form. In the experiment, two single strands of the first adapter were added separately. Wherein, the single strands of the 72-tagged sequence of the first strands (sequencing adapter single strands A) are added in the parallel form, i.e., the tag sequence of the first strand added to each parallel lane is the same. The single strands of the 72-tagged sequence of the second strands (sequencing adapter single strands B) are added in the transverse form, i.e., the tag sequence of the second strand added to each transverse lane is the same. And thus eventually forms a matrix of 72×72 tag sequences, each of which obtains a unique combination of a pairs of tags.

In order to achieve 72×72 tag sequence combinations using two separate tag sequences, the sequencing adapter single strands A with the tag sequences are added in a parallel form in the step of ligating adapters, and the sequencing adapter single strands B with the tag sequences are added in a transverse form, specifically as follows:

1. Adding the Sequencing Adapter Single Strands A

Preparation a reaction solution of the sequencing adapter single strands A as follows: 10× buffer (25% (volume ratio) PEG8000, 500 mM Tris-HCl, 10 mM ATP, 100 mM MaCl₂) 252 uL, adding nuclease free water to a total volume of 957 uL.

1) According to “72 Sample 35 nL Dispensing Process” of the wafergen MSND pipetting platform, it is required that the above-mentioned reaction solution of the sequencing adapter single strands A was added in 11.96 uL for each well to the above-mentioned parallel 72 wells of the 5184-well chip containing 300-1200 bp DNA fragment obtained in above second step;

2) Then according to “72 Sample 35 nL Dispensing Process” of the wafergen MSND pipetting platform, 72 different sequencing adapter single strands A (10 uM) were added in 2.14 uL for each well to the parallel 72 wells of the 5184-well chip after the treatment of step 1), centrifuge the liquid, and obtain a 5184-well plate with the sequencing adapter single strands A.

2. Adding the Sequencing Adapter Single Strands B

Preparation a reaction solution of the sequencing adapter single strands B as follows: 10× buffer (25% (volume ratio) PEG8000, 500 mM Tris-HCl, 10 mM ATP, 100 mM MaCl₂) 296.4 uL, T4 Ligase (600 U/uL, Enzymatics) 125.97 uL, adding nuclease free water to a total volume of 1186 uL.

1) According to “72 Sample 35 nL Dispensing Process” of the wafergen MSND pipetting platform, it is required that the above-mentioned reaction solution of the sequencing adapter single strands B was added in 13.95 uL for each well to the above-mentioned transverse 72 wells of the 5184-well chip with the sequencing adapter single strands A obtained in above first step;

2) Then according to “72 Sample 50 nL Dispensing Process” of the wafergen MSND pipetting platform, 72 different sequencing adapter single strands B (10 uM) were added in 1.65 uL for each well to the transverse 72 wells of the 5184-well chip after the treatment of step 1), centrifuge the liquid, and obtain a 5184-well plate with the sequencing adapter single strands B.

The above 5184-well plate with the sequencing adapter single strands B was subjected into a PCR instrument to react for 2 hours at 20° C., obtaining a 5184-well plate containing product of ligating the sequencing adapter.

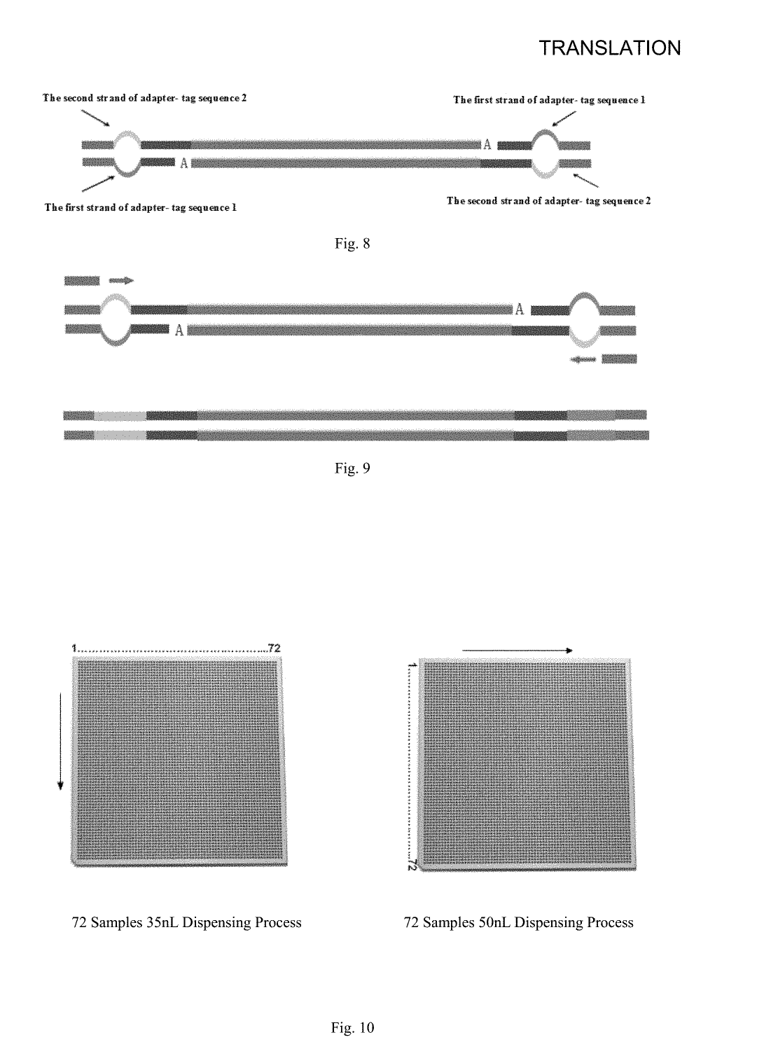

FIG. 8 is a schematic diagram of ligation two single strands of the first adapter (sequencing adapter) and an insert segment.

FIG. 10 shows two spray mode diagram for wafergen MSND. The left is the dispensing mode of the “72 Sample 35 nL Dispensing Process” cooperating with the sequencing adapter single strands A, a parallel form dispensing is achieved for the single strands (the sequencing adapter single strands A) with 72 tag sequences of the first strands. The right is the dispensing mode of the “72 Sample 50 nL Dispensing Process” cooperating with the second strands of the first adapters, a transverse form dispensing is achieved for the single strands (the sequencing adapter single strands B) with 72 tag sequences of the second strands.

Fourth, PCR amplification

FIG. 9 is a schematic diagram of the PCR amplification reaction after the ligation of the first adapter (the sequencing adapter).

The product of each well in the 5184-well plate containing product of ligating the sequencing adapter obtained in the third step, was taken out by centrifugation, and was mixed in a centrifuge tub, and then a PCR amplification is carried out, and during the amplification the dismatched portion of the adapters will be tuned to matched double strands. Because at the both ends of the same single strand of the product have two adapter single strands with different tag sequences, thus even the shape of the adapters change, the combination of the tag sequences will not be affected. And thus, the mixture of each well product will not affect the distinguishing of the combination of each tag sequence, specifically:

The product of each well in the 5184-well plate containing product of ligating the sequencing adapter obtained in the third step, was collected in a 1.5 mL centrifuge tub by centrifugation, and was subjected to purification by 1× AmpureXP beads. The purified product in 100 uL TE solution was collected, obtaining a product with sequencing adapter after purification. PCR amplification was carried out to the product, obtaining a PCR amplification product with sequencing adapter.

Primers used in the above PCR amplification are as follows:

The first strand of the PCR primers (upstream
primer):
(SEQ ID NO: 3)
GGUCGCCAGCCCUATGGC;
The second strand of the PCR primers (downstream
primer):
(SEQ ID NO: 4)
AGGGCUGGCGACCUTGTCAG.

The reaction system used in the above PCR amplification is as follows: product ligated to sequencing adapters 100 uL, 2× PfuCx buffer 275 uL, the first strand of the PCR primers (20 uM) 14 uL, the second strand of the PCR primers (20 uM) 14 uL, PfuCx polymerase 11 uL, adding nuclease free water to 550 uL.

PCR program of the above PCR amplification is as the following table 4.

### Table 4 is an Amplification Reaction Program

| Reaction temperature | Reaction time | | Number of cycles |
|---|---|---|---|
| 95° C. | 5 | min | |
| 95° C. | 30 | sec | 7 cycles |
| 56° C. | 30 | sec | |
| 72° C. | 4 | min | |
| 68° C. | 10 | min | |

| | |
|---|---|
| 10° C. | On hold |

The PCR amplification product of the product ligated to sequencing adapters was subjected to purification by 1× AmpureXP beads. And the purified product was dissolved in 60 uL TE solution.

FIG. 12 shows an electrophoretic gel of the product in each above step: 1, long fragment PCR amplification product (the amplification product obtained in the step 3) of the step 2 of the first step); 2, long fragment PCR negative amplification product (the amplification product obtained in the step 3) of the step 2 of the first step, water was used as the template instead of genomic DNA); 3, product removed of dUTP (step 1) of the second step); 4, negative product removed of dUTP (step 1) of the second step, the target fragment amplification product containing dUTP is replaced by water); 5, product with terminal A after gap translation (the polymerization product of the step 2) of the second step); 6, negative product with terminal A after gap translation (the polymerization product of the step 2) of the second step, the enzyme-digested product was replaced by water); 7, product 1 ligated to adapter (the product ligated to the sequencing adapter in the step 2 of the third step); 8, product 2 ligated to adapter (the product ligated to the sequencing adapter in the step 2 of the third step); 9, PCR product 1 (the PCR amplification product in the fourth step); 10, PCR product 2 (the PCR amplification product in the fourth step); 11, negative PCR product; M2, 100 bp maker. The target product of each step can be seen in FIG. 12.

Fifth, Digestion of dUTP of the amplification products

The PCR amplification product of the above-mentioned product ligated to the sequencing adapters needs to be digested the dUTP in the amplification product to form a sticky end, thereby achieving self-cyclization in subsequent experiments.

The dUTP digestion reaction was as follows: 10× Taq buffer 11 uL (RM00059, Complete Genomics), User enzyme (RM00017, Complete Genomics), purified PCR product 60 uL, adding nuclease free water to 110 uL, 37° C. for 1 hour to obtain reaction product.

Sixth, double strands cyclization

The reaction product obtained in the above fifth step was added to 10×TAbuffer (RM06601, Complete Genomics) 180 uL, adding nuclease free water to 1810 uL. The mixture was divided into 4 tubes on average. After 30 minutes in water bath at 60° C., the mixture was transferred to the room temperature bath for 20 minutes.

Preparing reaction mixture in advance, nuclease free water 98 uL, 20× Circ mix (MP01134, Complete, Genomics) 100 uL, Ligase (L603-HC-1500, Enzymatics) 2 uL, mixed together and divided into 4 portions and added into the above reaction product, and the reaction was carried out for 1 hour to cyclize to give a cyclized product.

The reaction product was recovered for each tube and purified using AmpureXP beads. The product was recovered in 70 μL TE.

Seventh, digestion the uncyclized DNA

In the cyclization reaction product, is in the presence of a considerable part of uncyclized DNA, in order not to affect the follow-up experimental results, the uncyclized DNA needs to be digested. Digestion reaction is as follows. For the purified cyclization product obtained from each of the above sixth step, adding 9×PS mix (MP01154, Complete Genomics) 8.9 uL, Plasmid-Safe (RM02046, Complete Genomics) 10.4 uL, and adding nuclease free water to 80 uL, after mixing, the reaction was carried out at 37° C. for 1 hour. The reaction product was purified using AmpureXP beads and dissolved in 40 uL of TE solution to give the product after the digestion.

Eighth, digesting and screening the target fragment using EcoP15

In the cyclized product, the adapters at both ends were ligated together, digesting with EcoP15 to cleave about 27 bp at the ends of the inserted fragment from the EcoP15 restriction site at both ends of the adapters, subsequently, screening the digested product to obtain a product with adapter sequence in the middle and the digested inserted fragment at both ends, specifically as follows:

Preparing the digestion system, the digested cyclized product obtained in the seventh step 37 uL, 5× EcoP 15 mix 3 (MP01149, Complete Genomics) 72 uL, EcoP 15 (RM00063, Complete Genomics) 10.8 uL, adding nuclease free water to 360 uL, and carried out a reaction at 37° C. for 16 hours.

Preparation of PEG32 beads (mixed by volume ratio, AmpureXP beads: 0.5% Tween=100: 1, the same below).

For the above 360 uL enzyme reaction product, 252 uL PEG32 beads were added and the supernatant was added to 184 uL PEG32 beads. After mixing, the beads were recovered and dissolved in a 52 uL TE recovery solution (with 0.001% Tween 20, by volume ratio), to obtain the digested product.

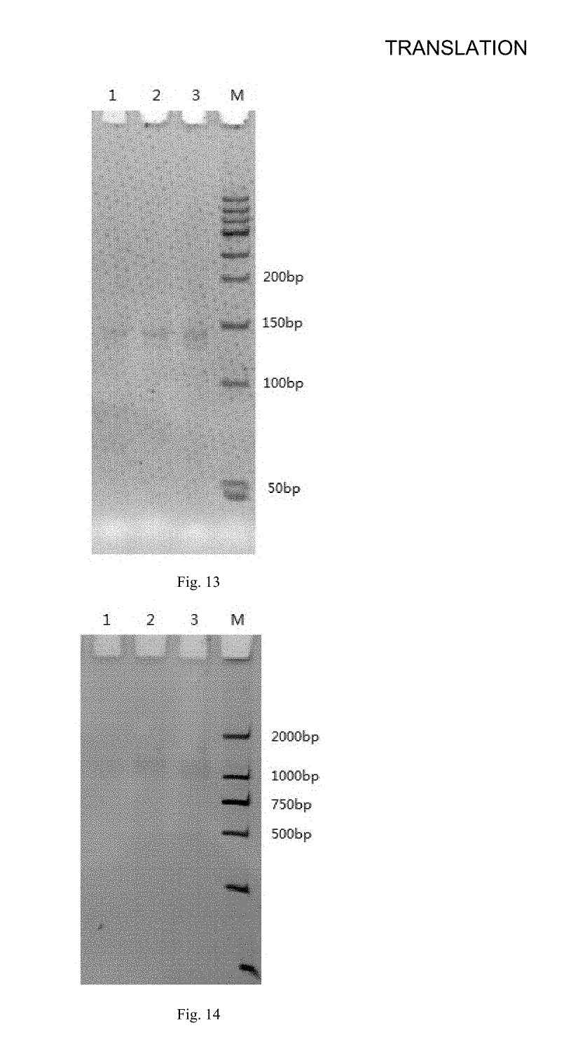

FIG. 13 is the product after EcoP15 endonuclease cleavage, and fragment recovery. The product fragment is around 140 bp.

Ninth, repairing the ends to blunt

The ends of the EcoP15 digestion product were repaired to blunt, in order to ligate the subsequent second adapter.

Preparation of the end-repairing reaction system, the digested products obtained in the above eighth step 44 uL, 10×NEB buffer 2 (New England biolabs) 5.4 uL, 25 mM dNTP 0.8 uL, 10 mg/mL BSA 0.4 uL, T4 DNA polymerase (M0203-com, New England Biolabs) at 12° C. for 20 min to give the end-repaired product.

The reaction product was purified using PEG32 beads and dissolved in 48 uL TE.

Tenth, dephosphorylation

The end-repaired product of the above-mentioned ninth step was subjected to a dephosphorylation treatment to be followed by a subsequent second adapter ligation. The reaction system was prepared as follows: 10×NEB buffer 2 (New England Biolabs) 5.75 uL, Fast AP (EF0651, Fermentas) 5.75 uL, the end-repaired and purified product 46 uL, at 37° C. for 45 minutes. The reaction product was purified using 75 uL PEG32 beads and dissolved in 42 uL TE solution to give the dephosphorylated product.

Eleventh, ligation of a second adapter

The second adapter was subjected to a directional ligation, and four steps of enzyme reactions were needed. Firstly, introduction of a 3′-terminal adapter sequence to the above dephosphorylated product in the tenth step, and again an end-repairing reactiong is performed and after introduction of dATP, introduced a 5′-terminal adapter sequence, and finally an oligonucleotide sequence was used to replace one of the sequences and ligated using a ligase.

Adapter sequences
3′-terminal adapter
/phos/ GTCTCCAGTCGAAGCCCGACG/3ddC/
/3ddC/AGAGGTCAGCTTCG
5′-terminal adapter
TTGGCCTCCGACT/3dT-Q/
/ddC/TGCTGGCGAACCGGAGGCTGA/5phos/
Two sequences of replacement
Oligonucleotide sequence 1
/52bio/TCCTAAGACCGCTTGGCCTCCGACT
Oligonucleotide sequence 2
/5phos/AGACAAGCTCGAGCTCGAGCGATCGGGCTTCGACTGGAGAC
PCR primers sequences
PCR primer 1
/52bio/TCCTAAGACCGCTTGGCCTCCGACT
PCR primer 2
/5phos/AGACAAGCTCGAGCTCGAGCGATCGGGCTTCGACTGGAGAC

Introduction of a Second Adapter 3′-Terminal Sequence

Preparation of reaction mixture, 3×HB (MP01139, Complete Genomics) 24.7 uL, nuclease free water 1.9 uL, T4 ligase (600 U/uL, Enzymatics) 1.9 uL, the second adapter 3′-terminal adapter 5.6 uL (9 uM), adding the dephosphorylated product 40 uL. The mixture was reacted at 14° C. for 2 hours and after the reaction, 63 uL PEG32 beads were used for purification and dissolved in 42 uL TE solution.

End-Repairing and Adding A

Preparation of the reaction mixture, 5× klex NTA mix (MP01150, Complete Genomics) 10.7 uL, klenow (RM00066, Complete Genomics) 1.1 uL, nuclease free water 1.5 uL, adding the product of the previous step 40 uL. The mixture was reacted at 37° C. for 1 hour. After the reaction, 69 uL PEG32 beads were used for purification and dissolved in 42 uL TE solution.

Introduction of a Second Adapter 3′-Terminal Sequence

Preparation of the reaction mixture, 3×HB 24.7 uL, nuclease free water 1.9 uL, T4 ligase 1.9 uL, the second adapter 3′-terminal adapter 5.6 uL (9 uM), adding the product of the previous step 42 uL. The mixture was reacted at 14° C. for 2 hours and after the reaction, 63 uL PEG32 beads were used for purification and dissolved in 42 uL TE solution.

Sequence Replacement

Preparation of Oligo reaction solution, 10× Taq buffer (RM00059, Complete Genomics) 8 uL, 100 mM ATP (MP01164, Complete Genomics) 0.8 uL, 25 mM dNTP 0.32 uL, oligonucleotide sequence 1 (20 mM) 1 uL, oligonucleotide sequence 2 (20 mM) 1 uL, and adding nuclease water to 32 uL.

Enzyme reaction solution, comprising 10× Taq buffer 0.4 uL, T4 ligase 4.8 uL, Taq polymerase 4.8 uL is prepared.

In the experiment, 32 uL Oligo reaction solution was added to 40 uL of the above purified DNA product, and then 8 uL of the enzyme reaction solution was added, mixed and incubated at 37° C. for 20 minutes. After the reaction, 80 uL PEG32 beads were used for purification and dissolved in 47 uL TE solution. The purified product was subjected to quantitative detection.

A second adapter ligation product was obtained.

Twelfth, amplification of the product ligated to the second adapter

2×PfuCx mix3 275 uL, PfuCx polymerase 11 uL, the second adapter PCR primer 1 (20 uM) 13.75 uL, the second adapter PCR primer 2 (20 uM) 13.75 uL, 60 ng of the product ligated to the second adapter after quantification in the above eleventh step were added into a 1.5 mL centrifuge tube, and added nuclease free water to 550 uL. After the reactants were mixed, they were averaged into four PCR tubes for PCR amplification reaction to obtain the amplified product.

The amplification reaction procedure is shown in Table 5 below.

### Table 5 is the Amplification Reaction Procedure

| Reaction temperature | Reaction time | | Number of cycles |
|---|---|---|---|
| 95° C. | 3 | min | |
| 95° C. | 30 | sec | 18 cycles |
| 60° C. | 30 | sec | |
| 72° C. | 4 | min | |
| 68° C. | 10 | min | |

Cooled to 4° C. at 0.1° C./s, on hold

After the reaction, 550 uL PEG32 beads were used for purification and dissolved in 85 uL TE solution. The purified product was quantitatively detected and 600 ng was subjected to subsequent single strand cyclization.

Thirteenth, single strand cyclization

1×BWB/tween mixture was prepared, 1×BWB (MP01111, Complete Genomics), 0.5% Tween20 20 uL was added to a centrifuge tube and mixed well.

Clean the beads. Took stretavidin beads (MP01162, Complete Genomics) 120 uL, placed in a magnetic frame to fully absorb the beads, removing the supernatant. Cleaned twice with 1×BBB (MP01110, Complete Genomics) 600 uL, then added 120 uL of 1×BBB to resuspend the beads and added 1% volume of 0.5% Tween20.

The product was subjected to single-strand separation using the cleaned beads. Took 600 ng of the amplified product from the above twelfth step, added TE to 60 uL, added 4×BBB (MP01145, Complete Genomics) 20 uL, and 30 uL stretavidin beads cleaned, mixed thoroughly and kept for 15 minutes, and then placed on a magnetic frame to fully adsorb the beads and then washed twice with a well prepared 1×BWB/tween mixture. After the completion of the cleaning, added 75 uL 0.1M NaOH solution, mixed for 2 minutes, placed on a magnetic frame to fully absorb the beads, recovering supernatant. Finally, to the recovered supernatant 0.3M MOPS acid (MP01165, Complete Genomics) 37.5 uL was added, and mixed evenly.

After completion of the above procedure, 112.5 uL of single-stranded product was obtained and the product was subjected to a single strand cyclization step. The following reaction solution was well prepared:

Primer mixture, Bridge Oligo (20 uM) 20 uL, adding 43 uL nuclease free water, mixing even;

Enzyme reaction mixture, 135.3 uL nuclease free water, adding 10×TA buffer (RM06601, Complete Genomics) 35 uL, 100 mM ATP 3.5 uL, ligase (600 U/uL) 1.2 uL, and the mixture was homogeneously mixed.

To the above 112.5 uL product, 63 uL of the primer mixture and 175 uL of the enzyme reaction mixture were mixed and homogenized and incubated at 37° C. for 1.5 hours to obtain a single strand cyclization product.

FIG. 14 shows the single strand cyclization electrophoregram.

Fourteenth, digestion of the uncyclized single strand products

To the single strand cyclization product obtained in the above thirteenth step, nuclease free water 1.5 uL, 10× TA buffer 3.7 uL, Exo I (M0293L, New England Biolabs) 11.1 uL, Exo III (M0206L, New England Biolabs) 3.7 uL were added, after mixing well, placed at 37° C. for 30 minutes. After completion of the reaction, 500 ml EDTA 15.4 uL was added, and the mixture was thoroughly mixed to terminate the reaction. The reaction mixture was purified by adding 500 uL of PEG32 beads and finally dissolved in 16 uL of TE solution to give a long fragment library.

The library preparation process ended.

### Example 2, Method for Library Construction (Adapted for Illumina Sequencing Platform)

First, long fragment DNA was interrupted into a 3-10 kb target fragment by a transposase

The same procedure as in Example 1 was carried out.

Second, the target fragment 3-10 KD target fragment was again fragmented into 300-1200 bp DNA short fragments

The same procedure as in Example 1 was carried out.

Third, ligation of sequencing adapters

Sequencing adapters with partial sequencing primers were ligated at both ends of the reaction product of the DNA fragment of 300-1200 bp size obtained in the above-mentioned second step, and the double strands of the adapters in this step had different tag sequences, respectively. In order to distinguish each well on the chip in the process of sequencing, in the chip in each parallel lanes (corresponding to the first strand of the sequencing adapter) adding the tag sequences numbered 1-72, and each transverse lanes (corresponding to the second strand of the sequencing adapter) adding the tag sequences numbered 1-72, so that a matrix of 72×72 is formed by two separate tag sequences so that each well has a unique combination of double tag sequences. Sequencing adapters with partial sequencing primers were ligated at both ends of the reaction product of the above step.

Sequencing adapter single strands A
(SEQ ID NO: 8)
AATGATACGGCGACCACCGAGATCTACACNNNNNNNNACACTCTTTC
CCTACACGACGCTCTTCCGATC*T

From the 5′ to 3′ direction, are in turn Flow cell adapter P5 (italic), 10 bases tag sequence (N) and a single-stranded DNA molecule complementary to Read 1 sequencing adapter (bold, * represents the phosphorothioate, that is, a nucleotide of phosphorothioate is used when the last nucleotide is synthesized).

Sequencing adapter single strands B
(SEQ ID NO: 9)
/5Phos/GATCGGAAGAGCACACGTCTGAACTCCAGTCACNNNNNN
NNATCTCGTATGCCGTCTTCTGCTTG /3′AmMO/

From the 5′ to 3′ direction, are in turn Read 2 sequencing adapter (bold), 10 bases tag sequence (N) and a single-stranded DNA molecule complementary to Flow cell adapter P7 (italic, Phos is phosphoric acid modification, AmMo is Amino-modification); the random fragments composed of 10 bases of the 72 3′ end tag primers are different from each other.

FIG. 7 shows a schematic diagram of adding two single strands with a tag respectively of the first adapter. In order to make each well in the chip with a unique tag sequence to distinguish between each other, the use of two tag sequences to form a combination form. In the experiment, two single strands of the first adapter were added separately. Wherein, the single strands of the 72-tagged sequence of the first strands are added in the parallel form, i.e., the tag sequence of the first strand added to each parallel lane is the same. The single strands of the 72-tagged sequence of the second strands are added in the transverse form, i.e., the tag sequence of the second strand added to each transverse lane is the same. And thus eventually forms a matrix of 72×72 tag sequences, each of which obtains a unique combination of a pairs of tags.

In order to achieve 72×72 tag sequence combinations using two separate tag sequences, the first adapter single strands with the tag sequences are added in a parallel form in the step of ligating adapters, and the second adapter single strands with the tag sequences are added in a transverse form, specifically as follows:

1. Adding the Sequencing Adapter Single Strands A

Preparation a reaction solution of the sequencing adapter single strands A as follows: 10× buffer (25% (volume ratio) PEG8000, 500 mM Tris-HCl, 10 mM ATP, 100 mM MaCl₂) 252 uL, adding nuclease free water to a total volume of 957 uL.

1) According to “72 Sample 35 nL Dispensing Process” of the wafergen MSND pipetting platform, it is required that the above-mentioned reaction solution of the sequencing adapter single strands A was added in 11.96 uL for each well to the above-mentioned parallel 72 wells of the 5184-well chip containing 300-1200 bp DNA fragment obtained in above second step;

2) Then according to “72 Sample 35 nL Dispensing Process” of the wafergen MSND pipetting platform, 72 different sequencing adapter single strands A (10 uM) were added in 2.14 uL for each well to the parallel 72 wells of the 5184-well chip after the treatment of step 1), centrifuge the liquid, and obtain a 5184-well plate with the sequencing adapter single strands A.

2. Adding the Sequencing Adapter Single Strands B

Preparation a reaction solution of the sequencing adapter single strands B as follows: 10× buffer (25% (volume ratio) PEG8000, 500 mM Tris-HCl, 10 mM ATP, 100 mM MaCl₂) 296.4 uL, T4 Ligase (600 U/uL, Enzymatics) 125.97 uL, adding nuclease free water to a total volume of 1186 uL.

1) According to “72 Sample 35 nL Dispensing Process” of the wafergen MSND pipetting platform, it is required that the above-mentioned reaction solution of the sequencing adapter single strands B was added in 13.95 uL for each well to the above-mentioned transverse 72 wells of the 5184-well chip with the sequencing adapter single strands A obtained in above first step;

2) Then according to “72 Sample 50 nL Dispensing Process” of the wafergen MSND pipetting platform, 72 different sequencing adapter single strands B (10 uM) were added in 1.65 uL for each well to the transverse 72 wells of the 5184-well chip after the treatment of step 1), centrifuge the liquid, and obtain a 5184-well plate with the sequencing adapter single strands B.

The above 5184-well plate with the sequencing adapter single strands B was subjected into a PCR instrument to react for 2 hours at 20° C., obtaining a 5184-well plate containing product of ligating the sequencing adapter.

FIG. 8 is a schematic diagram of ligation two single strands of the first adapter and an insert segment.

FIG. 10 shows two spray mode diagram for wafergen MSND. The left is the dispensing mode of the “72 Sample 35 nL Dispensing Process” cooperating with the first strand of the first adapter, a parallel form dispensing is achieved for the single strands with 72 tag sequences of the first strands. The right is the dispensing mode of the “72 Sample 50 nL Dispensing Process” cooperating with the second strands of the first adapters, a transverse form dispensing is achieved for the single strands with 72 tag sequences of the second strands.

Fourth, PCR amplification

FIG. 9 is a schematic diagram of the PCR amplification reaction after the ligation of the first adapter.

The product of each well in the 5184-well plate containing product of ligating the sequencing adapter obtained in the third step, was taken out by centrifugation, and was mixed in a centrifuge tub, and then a PCR amplification is carried out, and during the amplification the dismatched portion of the adapters will be tuned to matched double strands. Because at the both ends of the same single strand of the product have two different adapter single strands/different tag sequences, thus even the shape of the adapters change, the combination of the tag sequences will not be affected. And thus, the mixture of each well product will not affect the distinguishing of the combination of each tag sequence, specifically:

The product of each well in the 5184-well plate containing product of ligating the sequencing adapter obtained in the third step, was collected in a 1.5 mL centrifuge tub by centrifugation, and was subjected to purification by IX AmpureXP beads. The purified product in 100 uL TE solution was collected, obtaining a product with sequencing adapter after purification. PCR amplification was carried out to the product, obtaining a PCR amplification product with sequencing adapter.

Primers used in the above PCR amplification are as follows:

The first strand of the PCR primers (upstream
primer):
(SEQ ID NO: 10)
AATGATACGGCGACCACCGAGATCT;
The second strand of the PCR primers (downstream
primer):
(SEQ ID NO: 11)
CAAGCAGAAGACGGCATACGAGAT.

The reaction system used in the above PCR amplification is as follows: product ligated to sequencing adapters 100 uL, 2× PfuCx buffer 275 uL, the first strand of the PCR primers (20 uM) 14 uL, the second strand of the PCR primers (20 uM) 14 uL, PfuCx polymerase 11 uL, adding nuclease free water to 550 uL.

PCR program of the above PCR amplification is as the following table 6.

### Table 6 is an Amplification Reaction Program

| Reaction temperature | Reaction time | | Number of cycles |
|---|---|---|---|
| 95° C. | 5 | min | |
| 95° C. | 30 | sec | 7 cycles |
| 56° C. | 30 | sec | |
| 72° C. | 4 | min | |
| 68° C. | 10 | min | |

| | |
|---|---|
| 10° C. | On hold |

The PCR amplification product of the product ligated to sequencing adapters was subjected to purification by 1× AmpureXP beads. And the purified product was dissolved in 60 uL TE solution.

The reaction product of each step was subjected to electrophoretic detection to obtain the target product.

Library preparation is finished.

### Example 3, Analysis of Library Sequencing Results

First, sequencing

The DNA long fragment library prepared in Example 2 was sequenced on an illumina platform with a sequencing depth of 40×.

Second, comparison

Using the sequence alignment software SOAP aligner 2.20 (LiR, LiY, Kristiansen K, et al, SOAP: short oligonucleotide alignment program. Bioinformatics 2008, 24(5):713-714; LiR, YuC, LiY, et al, SOAP2: an improved ultrafast tool for short read alignment. Bioinformatics 2009, 25(15):1966-1967; http://soap.genomics.org.cn/soapaligner.html), the reads were alignmented to the human reference genome Reference hg 19 (http://hgdownload.cse.ucsc.edu/goldenPath/hg 19/bigZips/), only one comparison result (−r 1) is selected when there are multiple results.

Third, according to the tag combination information, to determine the corresponding reads to each well.

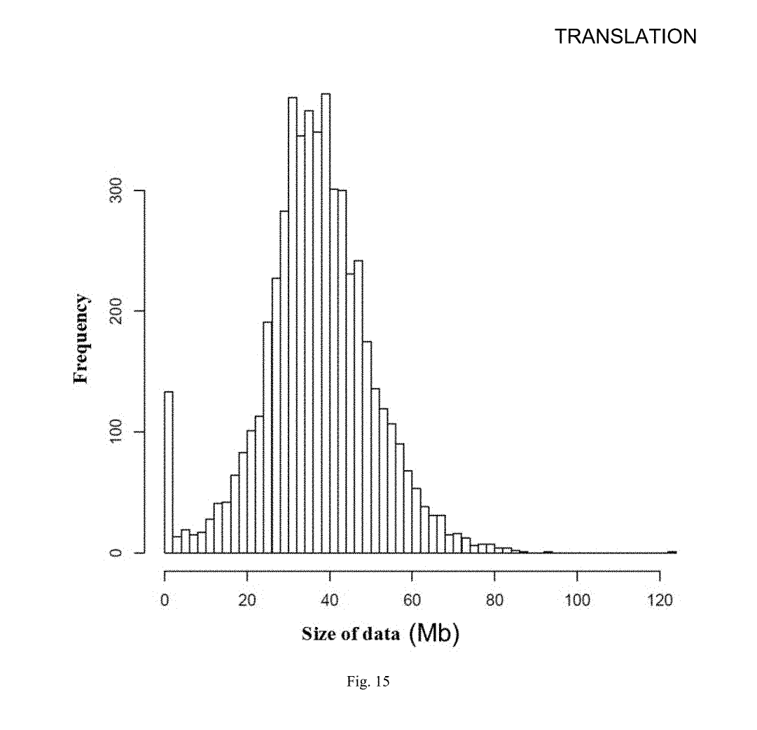

Fourth, statistics of the amount of data in each well and the corresponding frequency, draw the histogram (FIG. 15).

The statistics are as follows:

Unit: Mb

Mean: 36.9000; Standard deviation: 13.55647

Minimum: 0.3168; Maximum: 123.2000; Median: 36.7100

It can be seen from the above, this method can successfully mark all 5184 wells.

### Example 4: Comparison of Sequencing Results for Different Library Construction Methods

First, library construction and sequencing

A DNA long fragment library was prepared using a method for long fragment DNA library construction (Methods and compositions for long fragment read sequencing, U.S. Pat. No. 8,592,150) of CG company's Multiple Displace Amplification (MDA), and then on the Complete Genomics platform Sequencing is carried out, with a sequencing depth 80×.

A DNA long fragment library was prepared using Example 1 and then sequenced on a Complete Genomics platform with a sequencing depth of 60×.

Second, comparison

Using the sequence alignment software SOAP aligner 2.20 (LiR, LiY, Kristiansen K, et al, SOAP: short oligonucleotide alignment program. Bioinformatics 2008, 24(5):713-714; LiR, YuC, LiY, et al, SOAP2: an improved ultrafast tool for short read alignment. Bioinformatics 2009, 25(15):1966-1967; http://soap.genomics.org.cn/soapaligner.html), the reads were alignmented to the human reference genome Reference hg 19 (http://hgdownload.cse.ucsc.edu/goldenPath/hg 19/bigZips/), only one comparison result (−r 1) is selected when there are multiple results.

Third, calculating the single point coverage (−cvg) of Reads on the Reference by soap.coverage in SOAP aligner 2.20.

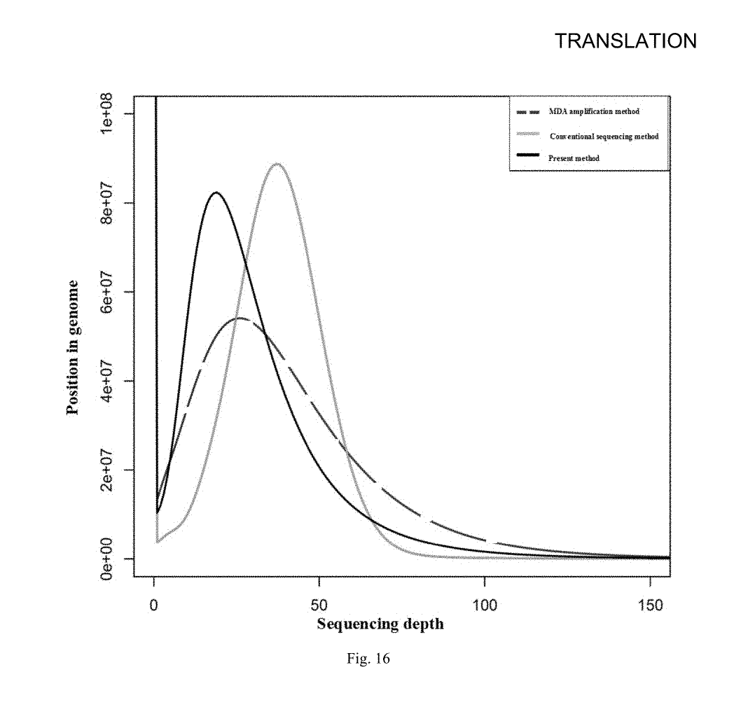

Fourth, drawing a distribution curve (FIG. 16) of the depth.

It can be seen from FIG. 16 that the amplification of this method is more homogeneous than the MDA amplification method and is closer to the sequencing depth distribution of the conventional sequencing method.

### INDUSTRIAL APPLICATIONS

The present invention is based on the principle of LFR library construction, replacing the method of MDA amplification using the original DNA amplification and the mode of the ligation of adapter A, and using the wafergen micro-pipetting platform instead of the 384-well plate to test. The present invention inserts a specific sequence by transposase and, performs PCR amplification of the DNA long fragment in each single well using this specific sequence. The amplification process is a conventional “denaturation-annealing-extension” mode, avoiding MDA amplification to form complex structures and non-specific amplification problems. In the present invention, two separate tag sequences are creatively designed in two single strands of a double-stranded adapter and then added separately in the form of a single strand, and two tag sequences are simultaneously ligated to both ends of the insert at the time of ligation. Since the ligation reaction is carried out at room temperature, the primers designed in the present invention are annealed in the normal temperature reaction system and then litigated to the both ends of the insert by the action of the ligase. The 3′ end of the interrupted insert is extended by a dATP prior to the ligation reaction, and the two single strands of the adapters are ligated at both ends of the insert through a terminal A-T pairing in the ligation reaction.

At the same time, the invention combines with the wafergen micro-pipetting platform to separate the DNA long fragment into a chip with 5184 wells, the number of single sample separation well is more than 10 times of the 384-well plate, the separation effect is more fully, thus obtaining better mutation location and assembling effect.

The experiment of the present invention demonstrates that the present invention improves the separation probability of homologous long fragments in the genome by combining with the microporous chip, and compared with the 384-well plate separation method of Complete Genomics, a better separation effect is achieved in the 5184 wells. In the separation of the 384-well plate, about 5% of the homologous long fragments will be divided into the same well and can not be distinguished, but in the 5184-well chip separation, the probability will be further reduced to improve the efficiency of analysis.

The invention amplifies the DNA fragments in each well after separation using the method of long fragment PCR, instead of MDA amplification in the original method, thus avoiding the formation of multi-level and complex structures of local area caused by MDA amplification. In the data, the data generated by these complex structures are removed because they can not be identified, limiting the resulting effective data ratio. Using the long fragment PCR amplification method, the obtained data is more efficient.

In the process of adapter ligation, both the adapter single strands are respectively with the tag sequences and added respectively. The ligation is carried out while annealing. Compared with the original method, adapter ligation-extension-ligation of the other adapter, it is possible to realize the ligation of different adapters at both ends in one reaction step, and introduce the combination of different tag sequences at both ends, which greatly reduces the complexity of the operation.

1. A method for constructing a long fragment DNA library, comprising the following steps:
1) A long fragment DNA is subjected to cleavage by transposase, amplification to introduce dUTP and then removing the dUTP to obtain cleaved fragments;
2) Sequencing adapter single strands A with different tags and sequencing adapter single strands B with different tags which are partially complementary thereto, are added respectively in single strand form into a system containing the cleaved fragments to generate a reaction, so that the cleaved fragments are ligated to sequencing adapters at both ends, to obtain products ligated to different sequencing adapters by the combination of the tags in the sequencing adapter single strands A and sequencing adapter single strands B, wherein the cleaved fragments are corresponding to different sequencing adapters from each other;
the sequencing adapter single strands A with different tags and the sequencing adapter single strands B with different tags can be annealed to form the sequencing adapters;
3) PCR amplification is carried out with DNA matched to the sequencing adapters as primers while using the product after the ligation of the sequencing adapters as a template, and the PCR amplification product obtained is a PCR amplification product ligated to different sequencing adapters;
4) Constructing a library using the PCR amplification product ligated to different sequencing adapters to obtain a long fragment DNA library.

2. The method of claim 1, wherein
the step 1) of the method comprises the steps of:
(1) cleaving the long fragment DNA with a transposase and ligating amplification adapters at both ends of the fragment after cleavage to obtain a product ligated to the amplification adapters;
(2) performing a first PCR amplification with the product ligated to the amplification adapters as a template and the DNA matched to the amplification adapters as primers to obtain a first PCR amplification product; and dUTP is incorporated during the first PCR amplification process;
(3) removing the dUTP in the first PCR amplification product, obtaining a gap, and performing a gap translation, and adding A at the 3′end, and obtaining the product of the cleaved fragments as step 1).

3. The method of claim 1, wherein
in the step 1), the transposase is a transposase embedding an amplification adapter;
and/or, the amplification adapter is one or two types, the amplification adapter being formed by a transposase-recognized single-stranded DNA molecule and a single-stranded DNA molecule partially reverse complementary thereto;
and/or, the size of the fragment between the two adjacent action sites of the transposase embedding the amplification adapter is 3-10 kb.

4. The method of claim 2, wherein
the amplification adapters are adapter 1 and adapter 2, wherein the adapter 1 is composed of a transposase-recognized single-stranded DNA molecule A and a single-stranded DNA molecule B partially reverse complementary thereto, and the adapter 2 is composed of the transposase-recognized single-stranded DNA molecule A and a single-stranded DNA molecule C partially reverse complementary thereto;
the DNA primers matched to the amplification adapters are composed of primer B and primer C, wherein the primer B is the remaining sequence of the single-stranded DNA molecule B excluding the portion complementary to the transposase-recognized single-stranded DNA molecule A; the primer C is the remaining sequence of the single-stranded DNA molecule C excluding the portion complementary to the transposase-recognized single-stranded DNA molecule A;
and/or, the method for removing the dUTP in the first PCR amplification product comprises the following:
the first PCR amplification product is subjected to an enzymatic cleavage reaction using uracil DNA glycosylase and human apurinic/apyrimidinic endonuclease to obtain a cleavage product; and then the digested product is subjected to a polymerization reaction using polymerase I, Taq polymerase and dATP to obtain a DNA fragment having a size of 300 to 1200 bp.

5. The method of claim 1, wherein
the tag sequences are obtained by arranging n bases, wherein the bases are at least one of A, G, C, and T, and n is equal to or greater than 8.

6. The method of claim 1, wherein
in the step 2), the sequencing adapter single strands A with different tags comprises a fragment A, a fragment B, a tag sequence and a fragment C in the direction of 5′ to 3′;
the sequencing adapter single strands B with different tags comprises a fragment reverse complementary to the fragment C, a tag sequence, a fragment D and a fragment reverse complementary to the fragment A in the direction of 5′ to 3′;
in the step 3), one primer is matched to the fragment B in the sequencing adapter single strands A; and the other primer is matched to the fragment D in the sequencing adapter single strands B.

7. The method of claim 1, wherein
the number of the sequencing adapter single strands A with different tags is less than or equal to 72, and the tag sequence of each single strand is different from each other;
the number of the sequencing adapter single strands B with different tags is less than or equal to 72, and the tag sequence of each single strand is different from each other;
n is greater than or equal to 8 and less than 15 in the tag sequence.

8. The method of claim 1, wherein
in the step 2), the sequencing adapter single strands A with tags and the sequencing adapter single strands B with different tags which are complementary thereto are added respectively in single strand form into a system containing the cleaved fragments to generate a reaction, comprises the following steps:
2)-A, 72 sequencing adapter single strands A with different tags are added respectively into 72 parallel lanes of a chip containing the cleaved fragments;
2)-B, 72 sequencing adapter single strands B with different tags are added respectively into 72 transverse lanes of the chip after the treatment of step 2)-A to react to generate a product ligated to the sequencing adapter.

9. The method of claim 1, wherein
in the step 3), one of the primers is identical to or reverse complementary to the fragment B in the sequencing adapter single strands A;
and the other primer is reverse complementary or identical to the fragment D in the sequencing adapter single strands B.

10. The method of claim 2, wherein
the step (2), (3) in step 1) and step 2) of the method are carried out in a 5184-well chip.

11. The method of claim 1, wherein
the long fragment DNA is a fragment of more than 100 kb, more specifically a fragment of 400 kb;
the transposase is a Tn5 transposase.

12. The method of claim 2, wherein
in the step (2), (3) of step 1) and step 2) of the method are carried out by wafergen MSND pipetting platform to add various substances to the wells of the chip.

13. The method of claim 12, wherein after adding various substances to the wells of the chip by wafergen MSND pipetting platform, the method further comprises the steps of centrifuging the substances.

14. The method of claim 2, wherein
the steps (1) and (2) of step 1) further comprise the steps of: digesting the transposase with a denaturing agent; the denaturing agent is specifically 0.1-1% SDS solution; and the digestion conditions are at 25° C. for 10 minutes.

15. The method of claim 4, wherein
in the step (1) of step 1), the conditions of the transposase cleavage reaction are at 55° C. for 5 minutes;
in the step (2) of step 1), the annealing temperature of the amplification is 68° C. and the annealing time is 18 minutes;
in the step (3) of step 1), the conditions of the digestion reaction are at 37° C. for 2 hours and then at 65° C. for 15 minutes; the conditions of the polymerization conditions are at 23° C. for 1 hour and then at 65° C. for 30 minutes;
in the step 2)-B of step 2), the reaction conditions are at 20° C. for 2 hours;
in the step 3), the annealing temperature of the PCR amplification is 68° C. and the annealing time is 10 min.

16. A long fragment DNA library prepared by method of claim 1.

17. A method for preparing a product ligated to a sequencing adapter, for the construction of a long fragment DNA library, wherein the method comprises the steps of: the step 1) to 2) of the method of claim 1.

18. A use of the method of claim 17 in the construction of a long fragment DNA library.


SEQUENCE LISTING (13 sequences)

SEQ ID NO: 1 - DNA, 57 bp, Artificial sequence
  Synthetic Sequence
  misc_feature (32)..(41): n is a, c, g, or t
cgaagcactcaaaggtcgccagccctatggcnnnnnnnnnncagtacgtcagcagtt

SEQ ID NO: 2 - DNA, 57 bp, Artificial sequence
  Synthetic Sequence
  misc_feature (16)..(25): n is a, c, g, or t
actgctgagtcgagannnnnnnnnnctgacaaggtcgccagccctgagtgcttcgaa

SEQ ID NO: 3 - DNA, 18 bp, Artificial sequence
  Synthetic Sequence
ggucgccagcccuatggc

SEQ ID NO: 4 - DNA, 20 bp, Artificial sequence
  Synthetic Sequence
agggcuggcgaccutgtcag

SEQ ID NO: 5 - DNA, 19 bp, Artificial sequence
  Synthetic Sequence
ctgtctcttatacacatct

SEQ ID NO: 6 - DNA, 33 bp, Artificial sequence
  Synthetic Sequence
tcgtcggcagcgtcagatgtgtataagagacag

SEQ ID NO: 7 - DNA, 34 bp, Artificial sequence
  Synthetic Sequence
gtctcgtgggctcggagatgtgtataagagacag

SEQ ID NO: 8 - DNA, 70 bp, Artificial sequence
  Synthetic Sequence
  misc_feature (30)..(37): n is a, c, g, or t
aatgatacggcgaccaccgagatctacacnnnnnnnnacactctttccctacacgacgctcttccgatct

SEQ ID NO: 9 - DNA, 65 bp, Artificial sequence
  Synthetic Sequence
  misc_feature (34)..(41): n is a, c, g, or t
gatcggaagagcacacgtctgaactccagtcacnnnnnnnnatctcgtatgccgtcttctgcttg

SEQ ID NO: 10 - DNA, 25 bp, Artificial sequence
  Synthetic Sequence
aatgatacggcgaccaccgagatct

SEQ ID NO: 11 - DNA, 24 bp, Artificial sequence
  Synthetic Sequence
caagcagaagacggcatacgagat

SEQ ID NO: 12 - DNA, 14 bp, Artificial sequence
  Synthetic Sequence
tcgtcggcagcgtc

SEQ ID NO: 13 - DNA, 15 bp, Artificial sequence
  Synthetic Sequence
gtctcgtgggctcgg